(19) United States
(12) Patent Application Publication
(10) Pub. No.: US 2025/0041568 A1
(43) Pub. Date: Feb. 6, 2025

(54) ECHOGENIC GUIDEWIRE

(71) Applicant: ABIOMED, Inc., Danvers, MA (US)

(72) Inventor: Christopher Sheils, Danvers, MA (US)

(73) Assignee: ABIOMED, Inc., Danvers, MA (US)

(21) Appl. No.: 18/788,899

(22) Filed: Jul. 30, 2024

Related U.S. Application Data
(60) Provisional application No. 63/529,856, filed on Jul. 31, 2023.

(51) Int. Cl. A61M 25/09

(52) U.S. Cl. CPC A61M 25/09041; A61M 2025/09058

(57) ABSTRACT

Disclosed is a guidewire for use in, e.g., procedures requiring axillary access and enhanced echo echogenicity. The guidewire may include a proximal portion and a distal portion. The distal portion may include a tapered core wire extending from the proximal portion to a distal tip. The distal portion may have surface texturing on at least one surface near a distal end of the distal portion. The guidewire may have, e.g., a 180° J-tip operably coupled to the tapered core wire. At least a portion of the J-tip may have a stiffness less than a stiffness of the tapered core wire. The J-tip may include a metal-filled polymer, such as a tungsten-filled polyurethane. The tapered core wire may have a maximum diameter of 0.6-0.7 mm, and may have a tapered length of 70-120 mm.

### CROSS-REFERENCE TO RELATED APPLICATIONS

The present application claims priority to U.S. Provisional Patent Application No. 63/529,856, filed Jul. 31, 2023, the contents of which are incorporated by reference herein in its entirety.

### TECHNICAL FIELD

The present disclosure is drawn to guidewires for medical procedures, and specifically, to an echogenic axillary guidewire.

### BACKGROUND

Axillary insertion of a blood pump to a patient offers unique challenges in surgical environments. Conventional techniques require a wire exchange procedure as typical guidewires cannot safely cross the aortic valve. This adds several minutes to a procedure, even if everything goes well on the first try. Insertion of the appropriate guidewire typically includes a substantial number of pausing to monitor wire and catheter position during the exchange.

Current commercially available guidewires are fluorogenic due to their density. However, most surgical environments do not include fluoroscopic instruments. Using guidewires outside of a dedicated catheterization lab or hybrid cath lab/operating room requires bringing in a portable C-arm for fluoroscopy, which has its own complications including footprint, staff requirements, etc. While current commercially available guidewires can reflect ultrasound, because guidewires are narrow their visibility via echocardiogram is limited. Getting a clear understanding of guidewire position in the left ventricle, for example, is difficult as the thin wire is rarely fully in plane with the ultrasound depth.

### BRIEF SUMMARY

In various aspects, a guidewire may be provided. The guidewire may include a proximal portion and a distal portion. The distal portion may include a tapered core wire extending from the proximal portion to a distal tip, the distal portion having a having surface texturing on at least one surface near a distal end of the distal portion. The guidewire may be free of a coil wire.

The guidewire may have, e.g., a 180° J-tip operably coupled to the tapered core wire. At least a portion of the 180° J-tip may have a stiffness less than a stiffness of the tapered core wire. The 180° J-tip may include a metal-filled polymer, such as a tungsten-filled polyurethane. The tapered core wire may include nitinol.

The guidewire may include a coating around at least a portion of the tapered core wire. The coating may include braided polytetrafluorethylene (PTFE).

The tapered core wire may have a circular cross-section across its entire length. The tapered core wire may have a maximum diameter of, e.g., 0.6-0.7 mm. The tapered core wire may have a minimum diameter of, e.g., 0.1 mm-0.2 mm. The tapered core wire may have a tapered length of 70 mm-120 mm. The guidewire may have a total length of 140 cm-160 cm.

In various aspects, a kit may be provided. The kit may include a guidewire as disclosed herein, and may include an introducer sheath and/or a closure device.

In various aspects, a method for delivering a medical device may be provided. The method may include providing a guidewire as disclosed herein. The method may include introducing the guidewire to the heart along a path through at least one blood vessel, the path crossing the aortic arch, where the guidewire is introduced to the heart without use of a guide catheter. The method may include slidably passing a medical device along the guidewire until the medical device is at a target location.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration showing a cut-away side view of a guidewire.

FIGS. 2A-2C are illustrations showing side-views of various distal tip extensions.

FIGS. 3A and 3B are illustrations showing cross-sectional views of grooves.

FIG. 4 is an illustration of a guidewire positioned within a heart.

FIG. 5 is an illustration showing a cut-away side view of a guidewire.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

In various aspects, a guidewire for delivering catheter-based devices with surfaces at or near the distal end treated to increase echogenicity may be provided. Surface texturing through chemical, electrochemical, or mechanical processes improves the reflection of ultrasonic waves so that regions of the wire are more echogenic and more visible on the echocardiogram, as has been explored with needles used to gain access for percutaneous interventions. This makes it easier for proper guidewire position to be determined under echocardiography without requiring fluoroscopy as is commonly used.

As mentioned above, this solves the problem of limited guidewire visibility under echocardiography like when echo is used to monitor blood pump (e.g., one of Abiomed's IMPELLA® blood pumps) delivery and positioning in, e.g., the left ventricle.

Current delivery guidewires are modeled after conventional 0.018″ diameter, 260 cm length wire designed for femoral access. This requires a wire exchange procedure as it cannot safely cross the aortic valve, which adds time and complexity to the procedure (e.g., 4-5 minutes, if everything proceeds well on the first attempt), with a substantial amount of pausing to monitor the wire and the catheter position during the exchange. To add some complexity, the wires are often difficult to see via transesophageal echocardiography, the most common imaging in operating rooms.

In addition to improving visibility during fluoroscopy, improved echogenicity may be desirable for better wire positioning, as some surgeons may not use fluoroscopy during delivery.

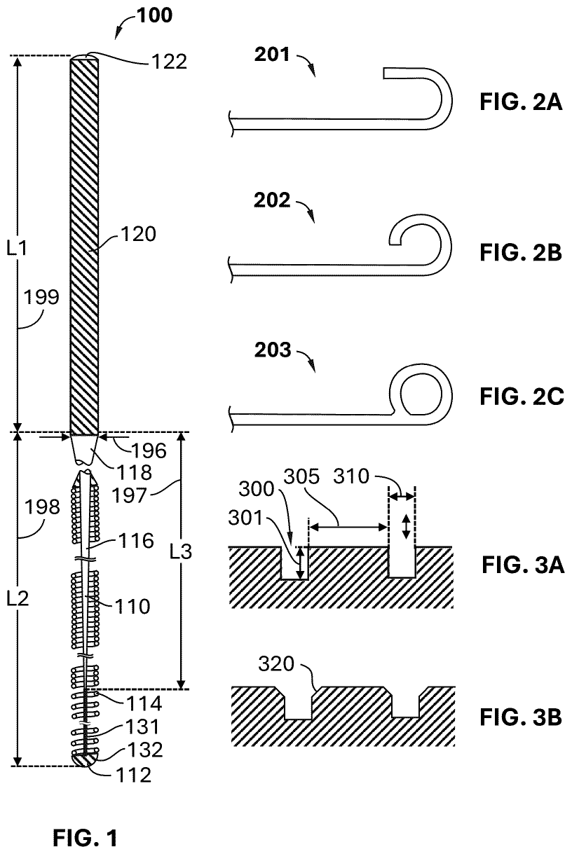

As seen in the FIG. 1, a guidewire (100) may include a distal section (110) that may include a distal tip (112), optionally a coil wire (114), a core wire (116), a transition section (118). The guidewire may include a proximal section (120) (e.g., a proximal section of the core wire (116)) having a proximal end (122).

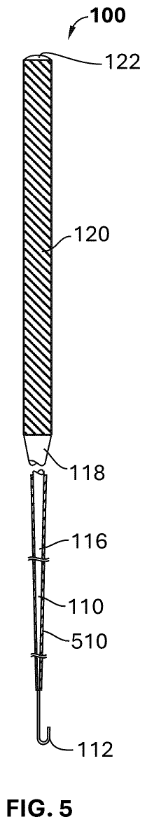

The coil wire (114), if utilized, may impart resistance to radial deformation and may allow the guidewire (100) to regain its original shape even after deformations that it may be subjected to during placement or manipulation in the heart. The coil wire (114) surrounds the core wire (116). Referring briefly to FIG. 5, in a preferred embodiment, the guidewire is free of a coil wire.

Referring to FIG. 1, the core wire (116) may have a diameter which decreases from the transition section (118) to the tip (112) of the distal section. The distal section (110) may have a length L2 (198).

The core wire (116) may also include a proximal section (120), extending between a proximal end (122) and the transition section (118). In some embodiments, the proximal section (120) may have a constant diameter. In some embodiments, the diameter of the proximal section may vary. The proximal section may be generally cylindrical, having a circular cross-section. The proximal section (120) may have a length L1 (199).

In some embodiments, L2 may be between 50-75% of the total length of the core wire (116). In some embodiments, L2 may be between 20-60% of the total length of the core wire (116). In some embodiments, L1 may be between 40-80% of the total length of the core wire (116). In some embodiments, L1 may be between 25-50% of the total length of the core wire (116).

The distal section (110) of the core wire may be more flexible than the proximal section of the guidewire (100). This allows a physician initially placing the guidewire into a patient to minimize damage to the arterial system of the patient.

Referring to FIGS. 2A-2C, in various embodiments, the distal tip (112) may include a distal extension. Such a distal extension may be, e.g., a 180° J-tip (201, FIG. 2A), a 270° tip (202, FIG. 2B), or even a closed loop tip (203, FIG. 2C).

In various embodiments, the tip (112) may be a platinum alloy. In various embodiments, the tip (such as a tip with a distal extension) may be atraumatic (e.g., relatively soft and flexible). In various embodiments, the tip may be a tungsten-filled polyurethane. The tip may have a stiffness gradient, with the highest stiffness near the core wire.

The guidewire (100) may include nitinol. For example, the core wire (116) may include nitinol.

At least some of the guidewire may include a coating. In some embodiments, the proximal section (e.g., proximal section (120)) may include a coating. In some embodiments, the core wire may include a coating. For example, as shown in FIG. 5, coating (510) may be disposed around some or all of the core wire (116). The coating may be, e.g., a braided polytetrafluorethylene (PTFE) coating. The coating may be a single color, or may be a multi-colored (such as a 2-colored) coating.

The taper core may have a circular cross-section, and may be free of a flattened end.

The length of the guidewire may be tailored based on the intended delivery approach. For example, the guidewire may have a total length that is no more than 250 cm. The guidewire may have a total length no more than 225 cm. The guidewire may have a total length no more than 200 cm. The guidewire may have a total length no more than 175 cm. The guidewire may have a total length no more than 160 cm. The guidewire may have a length of at least 125 cm. The guidewire may have a length of at least 140 cm. The guidewire may have a total length at least than 150 cm. The guidewire may have a total length between 125 and 175 cm.

The guidewire may have a taper length L3 (197) (e.g., the axial distance over which the guidewire tapers) that is no more than 150 mm. The guidewire may have a taper length no more than 140 mm. The guidewire may have a taper length no more than 130 mm. The guidewire may have a taper length no more than 120 mm. The guidewire may have a taper length no more than 110 mm. The guidewire may have a taper length no more than 100 mm. The guidewire may have a taper length of at least 70 mm. The guidewire may have a taper length of at least 80 mm. The guidewire may have a taper length of at least 90 mm. The guidewire may have a taper length of 70-120 mm.

The core wire may have a maximum diameter (196) that is no more than 0.8 mm. The core wire may have a maximum diameter no more than 0.75 mm. The core wire may have a maximum diameter no more than 0.7 mm. The core wire may have a maximum diameter no more than 0.65 mm. The core wire may have a maximum diameter of at least 0.6 mm. The core wire may have a maximum diameter of at least 0.55 mm.

The core wire may have a minimum diameter (e.g., the smallest diameter of the tapered of no more than 0.25 mm. The core wire may have a minimum diameter of at least 0.15 mm. The core wire may have a minimum diameter of at least 0.1 mm. The core wire may have a minimum diameter of no more than 0.25 mm. The core wire may have a minimum diameter of no more than 0.2 mm.

At least one surface at or near a distal end of the guidewire (such as an outer surface (131) of core wire (116) or an outer surface (132) of the distal tip (112)), may be surface textured.

For example, grooves in a surface can increase the intensity and angular range for echo visibility since the rate of reflection is improved from the otherwise convex surface. FIGS. 3A and 3B illustrate different examples of grooves. Referring to FIG. 3A, Grooves may be considered a portion of the guidewire that extends some depth (301) below adjacent surfaces on either side of the groove, each groove having a width (310).

In some embodiments, the distance (305) between grooves (300) may be the same. In some embodiments, the distance (305) between grooves may vary (e.g., at least one groove may have a spacing from an adjacent groove that is different than the spacing between two other grooves). In some embodiments, the spacing between each groove may be no more than 1 cm. In some embodiments, the spacing between each groove may be no more than 5 mm. In some embodiments, the spacing between each groove may be no more than 1 mm. In some embodiments, the spacing between each groove may be no more than 0.5 mm. In some embodiments, the spacing between each groove may be no more than 0.25 mm.

In some embodiments, the width (310) of each groove may be the same. In some embodiments, the width (310) of each groove may vary (e.g., at least one groove may have a width that is different from the width of another groove). In some embodiments, the width of each groove may be no more than 1 mm. In some embodiments, the width of each groove may be no more than 0.5 mm. In some embodiments, the width of each groove may be no more than 0.25 mm. In some embodiments, the width of each groove may be no more than 0.125 mm.

In some embodiments, the depth (301) of each groove may be the same. In some embodiments, the depth (301) of each groove may vary (e.g., at least one groove may have a depth that is different from the depth of another groove). In some embodiments, the depth of each groove may be less than 1 mm. In some embodiments, the depth of each groove may be no more than 0.5 mm. In some embodiments, the depth of each groove may be no more than 0.25 mm. In some embodiments, the depth of each groove may be no more than 0.125 mm. In some embodiments, the depth of each groove may be no more than 0.06 mm.

In some embodiments, the groove may have a rectangular cross-section, as shown in FIG. 3A. Referring to FIG. 3B, in some embodiments, one or more corners of a groove, such as a corner (320) connecting a groove to an adjacent outer surface, may be rounded or beveled. As will be envisioned, other cross-sectional types are envisioned, including “V” or “U” shapes, etc. In some embodiments, each groove has an identical cross-sectional type (e.g., all rectangular shapes, etc., even if the dimensions may vary). In some embodiments, one or more grooves may have a different cross-sectional type as compared to another groove (e.g., (i) some grooves may be rectangular with no bevels and some groove may be rectangular with bevels, or (ii) some grooves may be rectangular in shape, while other grooves are “V” shaped, etc.).

In some embodiments, a method may be provided. The method may include inserting an embodiment of a guidewire device as disclosed herein into a blood vessel of a subject via a lumen of an access device or introducer sheath. The method may include having the guidewire cross the aortic valve safely. The method may include attaching a medical device (such as a blood pump) to a proximal end of the guidewire device, and passing the medical device along the guidewire until the medical device into a desired location.

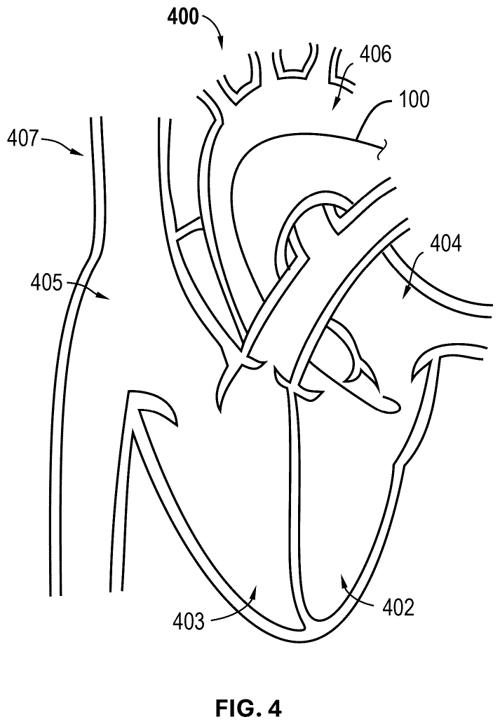

Referring to FIG. 4, a heart (400) can be seen. As seen, blood returning from the superior vena cava (407) to the heart passes through the right atrium (405) and into the right ventricle (403) before being sent to the lungs. Blood returning from the lungs enters the left atrium (404), then the left ventricle (402), before blood leaves the heart and passes into the aortic arch (406).

As noted, during some insertions, the guidewire may be configured to cross the aortic arch (406) in an atraumatic manner, and preferably without a guide catheter. Guide catheters are well known in the art; these typically include a tubular member having a lumen extending from a distal end to a proximal end, and the guidewire will typically extend through the guide catheter. Such guide catheters are often used to stabilize the position of the guidewire or other devices within the vessel or anatomical structure, helping to prevent them from shifting or causing damage. With the disclosed guidewires, one preferred configuration avoids the use of a guide catheter by having an appropriately designed stiffness profile, that is not too flexible or soft, as softer/more flexible wires may kink during insertion), but still flexible enough to cross the aortic arch atraumatically.

In some embodiments, a kit may be provided. The kit may include an embodiment of a guidewire device as disclosed herein. The kit may include an inflation device that may be removably attached to a proximal end of the guidewire device. In some embodiments, the kit may include an access device or introducer sheath which may have a lumen therethrough adapted to slidably receive the guidewire device. Such access devices are well known in the art, such as those disclosed in US 2023/0233802, as are introducer sheaths, such as those disclosed in U.S. Pat. No. 10,737,008 or U.S. Pat. No. 11,517,720. In some embodiments, the kit may include a closure device, such as a vascular closure device, such as an arterial closure device, which may have a lumen adapted to slidably receive the guidewire device. Such devices are well known in the art.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

What is claimed is:

1. A guidewire, comprising:
a proximal portion; and
a distal portion including a tapered core wire extending from the proximal portion to a distal tip, the distal portion having a having surface texturing on at least one surface near a distal end of the distal portion.

2. The guidewire of claim 1, wherein the guidewire is free of a coil wire.

3. The guidewire of claim 1, wherein the guidewire has a 180° J-tip operably coupled to the tapered core wire.

4. The guidewire of claim 3, wherein at least a portion of the 180° J-tip has a stiffness less than a stiffness of the tapered core wire.

5. The guidewire of claim 3, wherein the 180° J-tip comprises a metal-filled polymer.

6. The guidewire of claim 5, wherein the metal-filled polyurethane is a tungsten-filled polyurethane.

7. The guidewire of claim 1, wherein the tapered core wire comprises nitinol.

8. The guidewire of claim 7, wherein the guidewire includes a coating around at least a portion of the tapered core wire.

9. The guidewire of claim 8, wherein the coating comprises braided polytetrafluorethylene (PTFE).

10. The guidewire of claim 1, wherein the tapered core wire has a circular cross-section.

11. The guidewire of claim 1, wherein the tapered core wire has a maximum diameter of 0.6-0.7 mm.

12. The guidewire of claim 1, wherein the tapered core wire has a minimum diameter of 0.1 mm-0.2 mm.

13. The guidewire of claim 1, wherein the tapered core wire has a tapered length of 70 mm-120 mm.

14. The guidewire of claim 1, wherein the guidewire has a total length of 140 cm-160 cm.

15. A kit, comprising:
a guidewire of claim 1; and
an introducer sheath and/or a closure device.

16. A method for delivering a medical device, comprising:
providing a guidewire of claim 1;
introducing the guidewire to the heart along a path through at least one blood vessel, the path crossing the aortic arch, where the guidewire is introduced to the heart without use of a guide catheter.

17. The method of claim 1, further including slidably passing a medical device along the guidewire until the medical device is at a target location.
